(19) United States
(12) Patent Application Publication
(10) Pub. No.: US 2014/0341992 A1
(43) Pub. Date: Nov. 20, 2014

(54) COMPOSITIONS OF IMATINIB

(71) Applicant: HETERO RESEARCH FOUNDATION, Hyderabad (IN)

(72) Inventors: BANDI PARTHASARADHI REDDY, Hyderabad (IN); PODILI KHADGAPATHI, Hyderabad (IN); GOLI KAMALAKAR REDDY, Hyderabad, (IN); MADALLAPALLI KIRAN KUMAR, Hyderabad, (IN)

(21) Appl. No.: 14/224,803

(22) Filed: Mar. 25, 2014

(30) Foreign Application Priority Data
May 14, 2013 (IN) 2137/CHE/2013

(51) Int. Cl. A61K 31/506; A61K 9/28

(52) U.S. Cl. CPC A61K 31/506; A61K 9/2893; A61K 9/2866
USPC 424/468; 514/252.18

(57) ABSTRACT

The present invention relates to pharmaceutically acceptable compositions comprising imatinib, preferably imatinib mesylate with polymorphic stability and processes for the preparation thereof are disclosed.

### CROSS-REFERENCE TO RELATED APPLICATIONS

This patent application claims priority to Indian patent application number 2137/CHE/2013, filed on May 14, 2013, the contents of which are incorporated by reference herein in their entirety.

### FIELD OF THE DISCLOSURE

The present disclosure relates to pharmaceutically acceptable compositions comprising imatinib, more specifically film-coated tablets comprising imatinib mesylate.

### BACKGROUND

Imatinib mesylate, 4-[(4-Methyl1-piperazinyl)methyl]-N-[4-methyl-3-[[4-(3-pyridinyl)-2-pyrimidinyl]amino]-phenyl]benzamide methanesulfonate, disclosed in EP0564409. Its structural formula as follows:

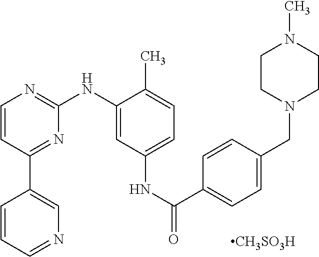

Imatinib mesylate is marketed under the trade name Glivec® in Europe by Novartis in the form of 100 mg and 400 mg tablets for the treatment of various cancers including dermatofibrosarcoma, gastrointestinal stromal tumors and chronic myelogenous leukemia.

Several crystalline polymorphic forms of imatinib mesylate are known. For example, international patent application WO9903854 discloses alpha and beta crystalline forms of Imatinib mesylate. According to WO9903854, beta crystalline form of Imatinib mesylate is more suitable for pharmaceutical applications than alpha form due to the improved stability and flow characteristics of beta form.

For sake of treatment compliance and patient convenience, high drug loaded tablets with a decreased size have been developed.

For instance, International patent application WO2003090720 discloses film coated tablets prepared by wet granulation comprising from 30% to 80% by weight of imatinib mesylate. Exemplified tablets are coated with aqueous coating dispersion. According to the preferred embodiments disclosed, imatinib mesylate is present in the tablets as beta crystalline form. Further, WO11160798A discloses film-coated tablets with an amount of imatinib mesylate between 82-89% which has been wet granulated in water without any further excipient.

Additionally, the anonymous document, “stable tablet formulation containing more than 80% of Imatinib mesylate” IP.COM, reference IPCOM000167554D, 2008, discloses tablets comprising 85% of imatinib mesylate manufactured either by wet granulation, direct compression or roller compaction and coated using a hypromellose based film coat. This document is silent about the crystalline form of imatinib mesylate used in the formulation and the coating solvent.

High drug loaded tablets containing imatinib mesylate in alpha crystalline form have been also disclosed. For example, International patent application WO2012019633 discloses tablets comprising around 60% of alpha imatinib mesylate, compressed from a granulate containing 95-99% of the active ingredient prepared by wet granulation, and coated with an aqueous film-coating solution. Additionally, US2007036850A discloses tablets comprising 25-80% of imatinib mesylate, preferably in the alpha form, made by dry granulation and coated with an aqueous film-forming solution.

International Publication WO2011121593A1 discloses high drug loaded film-coated tablet compositions containing imatinib mesylate in amounts exceeding 80%, wherein the tablets were coated with aqueous-based film coating comprising polyvinyl alcohol.

The prior art imatinib high loaded tablet compositions show stability problems when subjected to stress conditions, e.g., polymorphic conversion issues.

Hence, there is a need to develop high drug loaded tablets of imatinib with high stability, particularly which are stable under stress conditions, e.g. stable against polymorphic conversion.

### SUMMARY

The invention provides high drug loaded film coated tablet compositions comprising imatinib mesylate, more in particular in alpha crystalline or amorphous form, wherein the initial solid form of imatinib mesylate is retained upon storage (e.g., during 1 to 3 months at 40° C.±2° C. and 75%±5% relative humidity).

The compositions described herein have unexpectedly shown improved stability over prior art high loaded imatinib tablets, in particular e.g. over those compositions film-coated with aqueous film coating solutions.

Without wishing to be bound by theory, the increased stability may be attributed to the film-coating carried out with organic solvent or mixture of organic solvents, preferably comprising hydroxypropyl methylcellulose.

Thus, the inventors have surprisingly found that coating the core of a high drug loaded tablet of alpha imatinib mesylate with a film-forming coating solution in an organic solvent prevents alpha imatinib mesylate from undergoing polymorphic changes upon storage even under stress conditions.

The compositions described herein have shown other improvements over prior art compositions, e.g. improved dissolution rate.

Therefore, the first aspect of the present invention is directed to an immediate release film coated tablet comprising 80% to 99% by weight of imatinib mesylate salt based on the total weight of the film-coated tablet, wherein the tablet core comprising the active ingredient and one or more pharmaceutically acceptable excipients, is coated with a solution or dispersion in an organic solvent or mixture of organic solvents of a film coating agent comprising hydroxypropyl methylcellulose.

The second aspect of the invention is directed to a process for preparing an immediate release film coated tablet comprising 80% to 99% by weight of imatinib mesylate salt based on total weight of the film coated tablet and one or more pharmaceutically acceptable excipients, comprises coating a tablet core comprising the active ingredient and one or more pharmaceutically acceptable excipients with a solution or dispersion in an organic solvent or mixture of organic solvents of a film coating agent comprising hydroxypropyl methylcellulose.

Furthermore, the invention also relates to the product obtained by the process according to the second aspect of the invention.

### DETAILED DESCRIPTION

The crystalline form alpha of imatinib mesylate is a polymorphic form as prepared and characterized by methods disclosed in WO9903854, more specifically as shown in Example 1 of WO9903854.

Alpha crystalline form of imatinib mesylate is characterized by a powder x-ray diffraction pattern showing its relevant peaks at 2θ angle values of about 4.9, 10.5, 14.9, 16.5, 17.7, 18.1, 18.6, 19.1, 21.3, 21.6, 23.2, 24.9, 28.0, and 28.6±0.2 degrees.

An immediate-release dosage form, as defined in European Pharmacopoeia 7.0 Apr. 2010:1502, is a preparation showing a release of the active substance which is not deliberately modified by a special formulation design and/or manufacturing method. In the case of a solid dosage form, the dissolution profile of the active substance depends essentially on its intrinsic properties.

By an organic solvent based film coating is meant herein a film coating that is applied employing a coating solvent system consisting of an organic solvent or mixture of organic solvents.

Organic solvent or mixture of organic solvents as described herein may contain up to a 10% (w/w) of water, preferably the solvents are anhydrous. By anhydrous organic solvent it is mean herein an organic solvent that contain less than 5% (w/w) of water.

The immediate release film coated tablet of the invention preferably comprises imatinib mesylate in alpha crystalline form. Advantageously, in said immediate release film coated tablet, said alpha crystalline form of imatinib mesylate is retained upon storage at 40° C. and at 75% relative humidity for 3 months, when measured by XRPD technique.

In another embodiment, the immediate release film coated tablet of the invention comprises imatinib mesylate in amorphous form. Most preferably, in said immediate release film coated tablet, imatinib salt does not undergo polymorphic conversion upon storage under stress conditions.

Preferably, the immediate release film coating agent used in the compositions of the invention comprises hydroxypropyl methylcellulose, for example hydroxypropyl methylcellulose based Opadry® product range and mixtures of hydroxypropyl methylcellulose and polyethylene glycol. More preferably, the film coating agent is an Opadry® composition comprising hydroxypropyl methylcellulose, polyethylene glycol/macrogol and talc.

According to the present invention, the core tablet may comprise pharmaceutically acceptable excipients selected from diluents, glidants, lubricants and combinations thereof. The expression “pharmaceutically acceptable excipients” or simply “excipients” refers to pharmaceutically acceptable materials, compositions or vehicles. Each component must be pharmaceutically acceptable in the sense of being compatible with the other ingredients of the pharmaceutical composition. It must also be suitable for use in contact with the tissue or organ of humans and animals without excessive toxicity, irritation, allergic response, immunogenicity or other problems or complications commensurate with a reasonable benefit/risk ratio.

Exemplary diluents include microcrystalline cellulose, lactose, starch, pregelatinized starch, calcium carbonate, calcium sulfate, sugar, dextrates, dextrin, dextrose, dibasic calcium phosphate, tribasic calcium phosphate, kaolin, magnesium carbonate, magnesium oxide, maltodextrin, mannitol, polymethacrylates, potassium chloride, powdered cellulose, sodium chloride, sorbitol, talc and the like, and combinations thereof.

Exemplary binders include polyvinyl pyrrolidone, copovidone, hydroxypropyl cellulose, hydroxypropyl methylcellulose, pregelatinized starch, powdered acacia, gelatin, guar gum, carbomers and the like, and combinations thereof.

Exemplary glidants include colloidal silicon dioxide, magnesium trisilicate, powdered cellulose, starch, talc, tribasic calcium phosphate and the like, and combinations thereof.

Exemplary lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, zinc stearate and the like, and combinations thereof. Preferred lubricant is magnesium stearate.

Advantageously, the process of the inventions provide small tablets with a high load of drug, maintaining the amount of excipients as low as possible while meeting the requirements of bioavailability and bioequivalence of pharmaceutical products. In a preferred embodiment, the tablet core of the compositions of the invention, obtained by the process of the invention, consists of imatinib mesylate and lubricants, more preferably magnesium stearate.

In one embodiment the tablet core consists of 0.10-1.00% of magnesium stearate and 97.75-99% of imatinib mesylate, and 0.75-1.25% of HPMC based film-coating by weight percentage of the final film-coated tablet.

In a further preferred embodiment the tablet core consists of 97.75-99.00% of imatinib mesylate, 12.00%-16.00% of diluent(s), 0.10-1.00% of magnesium stearate and 0.75-1.25% of HPMC based film-coating by weight percentage of the final film-coated tablet.

For the purposes of the present invention, unless otherwise stated, the term “percentage (%) by weight” refers to the percentage of each ingredient of the composition in relation to the total weight of the final film-coated tablet.

For the purposes of the invention, any ranges given include both the lower and the upper end-points of the range. Ranges given should be considered approximate, unless specifically stated.

According to the process of the invention, the coating process of the tablet core is carried out in an organic solvent or a mixture of organic solvents.

In a preferred embodiment, the organic solvent or mixture of organic solvents used in the process of the invention is selected from C1-C3 alcohol, e.g. methanol, ethanol or isopropyl alcohol, ethyl acetate, ethyl lactate, acetone, methylene chloride, 1,1,1-trichloroethane, chloroform, and mixtures thereof. Preferably, the solvent system is a mixture of C1-C3 alcohol/methylene chloride, more preferably Isopropyl alcohol/methylene chloride.

In a preferred embodiment of the invention, the solvent system of the coating step is anhydrous, i.e., it has a content of water up to 5% in weight of the solvent system.

The coating step of the process of the invention encompasses applying the coating solution to the core tablet and drying the coated tablet for removing the solvent and obtaining the final film-coated tablet by processes and methods known in the art. The appropriate conditions and/or equipment used, can readily be determined by those skilled in the art according to the tablet being prepared.

Similarly, tablet core compositions of the present invention can also be prepared according to methods well known in the state of the art. Most preferred method for preparing the tablet core of the compositions of the invention is by direct compression.

In a particularly preferred embodiment of the process of the invention, the tablet core is prepared by direct compression of imatinib mesylate, optionally together with one or more pharmaceutically acceptable excipients or carriers for preparing the tablet core. In a still more preferred embodiment, the tablet core is prepared by direct compression of alpha imatinib mesylate, a lubricant and optionally one or more excipients. Most preferably imatinib is either in alpha form or amorphous form.

Direct compression involves blending a composition comprising imatinib mesylate as crystalline alpha form and one or more excipients and compressing it directly into a tablet. Direct compression is easy, simple and applicable for industrial scale. The appropriate conditions and/or equipment used for suitable compressing the blend for obtaining the core tablet can readily be determined by those skilled in the art according to the tablet to be prepared.

With regard to the specific conditions for carrying out the processes of the invention, the skilled person would know how to adjust the parameters of each of the steps indicated above in the light of the description and examples of the present invention.

As defined by the third aspect, the invention also encompasses an immediate-release film-coated tablet obtained by the process of the invention.

The immediate-release film-coated tablet “obtained by” the process of the invention is used here to define the composition by the process for obtaining it and refers to the product obtained by the preparation process comprising the coating step as defined above. For the purposes of the invention the expressions “obtainable”, “obtained” and equivalent expressions are used interchangeably, and in any case, the expression “obtained” encompasses the expression “obtainable”.

All the embodiments of the immediate-release film-coated tablet of the invention contemplate all the combinations provided by all the embodiments of the process of the invention and the combinations thereof.

Further, the invention also encompasses an immediate-release film-coated tablet comprising from 95 to 99% w/w of imatinib mesylate salt, based on the total weight of the film-coated tablet, and one or more pharmaceutically acceptable excipients, wherein said mesylate salt is in alpha crystalline form and the tablet is prepared by a process comprises coating a core tablet with a film coating agent employing an organic solvent or mixture of organic solvents as coating solvent system.

The immediate-release film-coated tablet obtained by the process of the invention has surprisingly shown improved storage stability maintaining imatinib mesylate stable in crystalline alpha form without undergoing polymorphic conversion upon storage.

Polymorphic conversion is measured by techniques known in the art. Preferably, the percentage of polymorphic conversion is measured by known PXRD techniques. For instance, in a mixture of polymorphs the amount of each polymorph can be calculated with reference to the relative intensity of the unique characterizing XRD peaks of each polymorph.

In a more preferred embodiment of the invention, the immediate-release film-coated tablet obtained by the process of the invention retains alpha crystalline form of imatinib mesylate upon storage at 40° C., 75% RH, for 3 months.

A further aspect of the invention is directed to a composition comprising a tablet core obtained by direct compression of a composition that comprises 95% to 99% of imatinib mesylate based on total weight of the film coated tablet composition and one or more excipients, wherein the tablet core is coated with an organic solvent film coating solution comprising hydroxypropyl methylcellulose.

In yet further aspect of the invention is directed to a composition comprising a tablet core comprises 80% to 99% of imatinib mesylate based on total weight of the film coated tablet composition and one or more excipients, wherein the tablet core is coated with an organic solvent film coating solution comprising hydroxypropyl methylcellulose.

A further aspect of the invention is directed to a stable film-coated immediate-release tablet composition comprising alpha crystalline form of imatinib mesylate and a pharmaceutically acceptable excipient, wherein the tablet composition retains its initial polymorphic form upon storage (e.g. during 1 to 3 months at 40° C.±2° C. and 75%±5% relative humidity). More specifically, wherein said film coating is applied as an organic solvent based film coating comprising hydroxypropyl methylcellulose.

A further aspect of the invention is directed to a stable film coated tablet composition comprising, based on total weight of the film coated tablet, 97 to 99% of alpha crystalline form of imatinib mesylate, 0.1 to 1% of magnesium stearate and 0.75 to 1.25% of hydroxypropyl methylcellulose based film coating; and the composition is prepared by direct compression process. Preferably, wherein the film coating is an organic solvent based film coating.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### EXAMPLES

Following examples are given for the purpose of illustrating the invention and shall not be construed as limiting the scope of the invention.

### Example 1 to 4

TABLE 1

| | Example 1 | Example 2 | Example 3 | Example 4 |
|---|---|---|---|---|
| Ingredients | mg/tablet | mg/tablet | mg/tablet | mg/tablet |
| Imatinib mesylate | 478.00^{#} | 478.00^{#} | 478.00^{#} | 717.00^{@} |
| (α-form) | | | | |
| Magnesium stearate | 0.75 | 2.00 | 3.00 | 6.00 |
| Coating: | | | | |
| Opadry ® yellow | 4.78 | 4.80 | 4.81 | 7.23 |
| 03F82597* | | | | |
| Iso propyl alcohol | q.s. | q.s. | q.s. | q.s. |
| Methylene chloride | q.s. | q.s. | q.s. | q.s. |
| Coated tablet weight | 483.53 | 484.80 | 485.81 | 730.23 |

^{#}Each 478 mg of Imatinib mesylate contains 400 mg of Imatinib.
^{@}Each 717 mg of Imatinib mesylate contains 600 mg of Imatinib.
*Opadry ® yellow 03F82597 composition comprises hydroxypropyl methylcellulose, iron oxide yellow, polyethylene glycol/macrogol, talc, titanium dioxide and iron oxide red.

Brief Manufacturing Process:
1. Imatinib mesylate was sifted and loaded into blender,
2. magnesium stearate was sifted and loaded into blender and blended for 5 minutes,
3. lubricated blend of step 2 was compressed into tablet,
4. tablet of step 3, was film coated using Opadry® yellow 03F82597 dispersion in isopropyl alcohol and methylene chloride.

### Example 5

### Comparative Example

Comparative dissolution study was conducted between the composition of imatinib prepared according the present invention and composition of imatinib prepared according to the International Publication WO2011121593A1:

### Dissolution Conditions:

### Dissolution Medium: 0.1N HCl

### Volume: 1000 ml

### Apparatus: USP II (Paddle)

### Speed: 50 RPM

TABLE 2

| | Composition of imatinib | Composition of |
|---|---|---|
| Time | prepared according the | imatinib prepared |
| in | present invention | according to the |
| minutes | (Example 3) | ′593 patent |
| 10 | 58 | 54 |
| 15 | 82 | 73 |
| 20 | 94 | 85 |
| 30 | 102 | 98 |
| 45 | 103 | 102 |

As can be seen from Table 2, composition of imatinib prepared as per Example 3 of the present invention showed slightly better dissolution properties as compared to composition of imatinib prepared according to the '593 patent.

### Example 6

### Stability Study

Stability of imatinib tablets prepared as per Example 3 were evaluated based on polymorphic stability after storage for three months at 40° C. and 75% relative humidity.

The polymorphic stability refers to the stability of alpha crystalline form of imatinib mesylate to remain in the initial polymorphic form without undergoing polymorphic conversion after storage.

The polymorphic conversion is measured by techniques known in the art. In particular, each known polymorphic form of imatinib may be characterized by a unique set of peaks in the powder x-ray diffraction pattern having 2θ angle positions. Table 3 below shows powder x-ray diffraction pattern of Imatinib tablets (Example 3) stored at 40° C. and 75% RH for 3 months

Results from Table 3, showed that the alpha crystalline form of imatinib mesylate is retained in the tablets that were coated with organic solvent based hydroxypropyl methylcellulose.

TABLE 3
Peaks in the powder x-ray diffraction pattern (XRD)

| Initial form of imatinib | After storage for three |
|---|---|
| mesylate, having 2θ angle | months, having 2θ angle |
| positions at about | positions at about |
| (±0.2 degrees) | (±0.2 degrees) |
| 4.914 | 4.951 |
| 10.459 | 10.501 |
| 14.913 | 14.949 |
| 16.511 | 16.554 |
| 17.708 | 17.765 |
| 18.107 | 18.164 |
| 18.644 | 18.695 |
| 19.099 | 19.147 |
| 21.298 | 21.348 |
| 21.640 | 21.690 |
| 23.184 | 23.222 |
| 24.911 | 24.941 |
| 28.050 | 28.071 |
| 28.568 | 28.592 |

### Example 7 to 8

TABLE 4

| | Example 7 | Example 8 |
|---|---|---|
| Ingredients | mg/tablet | mg/tablet |
| Imatinib mesylate^{#} (amorphous) | 478.00 | 478.00 |
| Lactose monohydrate | 40.00 | — |
| Microcrystalline cellulose | 37.00 | — |
| Povidone K-90 | 5.00 | — |
| Colloidal silicon dioxide | 2.00 | — |
| Magnesium stearate | 5.00 | 3.00 |
| Coating: | | |
| Opadry ® yellow 03F82597 | 8.00 | 4.81 |
| Iso propyl alcohol | q.s. | q.s. |
| Methylene chloride | q.s. | q.s. |
| Coated tablet weight | 575.00 | 485.81 |

^{#}Each 478 mg of Imatinib mesylate contains 400 mg of Imatinib.

Brief Manufacturing Process:

1. All the ingredients except magnesium stearate were sifted and loaded into blender,

2. magnesium stearate was sifted,

3. blend of step 1, was lubricated with magnesium stearate of step 2,

4. lubricated blend of step 3, was compressed into tablet,

5. tablet of step 4, was film coated using Opadry® yellow 03F82597 dispersion in isopropyl alcohol and methylene chloride.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. An immediate release film coated tablet comprising
a tablet core comprising imtainib mesylate and one or more pharmaceutically acceptable excipients, and
a coating disposed on the tablet core, wherein the coating is applied from a solution or dispersion of hydroxypropyl methylcellulose in an organic solvent or mixture of organic solvents;
wherein the film coated tablet contains 95% to 99% by weight of imatinib mesylate based on the total weight of the film-coated tablet.

2. The immediate release film coated tablet of claim 1, wherein said imatinib mesylte is in alpha crystalline form characterized by a powder X-ray diffraction (PXRD) pattern having peaks at following 2θ angle positions at about 4.9, 10.5, 14.9, 16.5, 17.7, 18.1, 18.6, 19.1, 21.3, 21.6, 23.2, 24.9, 28.0, and 28.6±0.2 degrees.

3. The immediate release film coated tablet of claim 2, wherein said alpha crystalline form of imatinib mesylate is retained upon storage at 40° C. and at 75% relative humidity for 3 months, when measured by an XRPD technique.

4. The immediate release film coated tablet of claim 1, wherein said mesylate salt is in amorphous form.

5. A process for preparing an immediate release film coated tablet, comprising
providing a tablet core comprising imatinib mesylate and one or more pharmaceutically acceptable excipients, and
coating the tablet core with a solution or dispersion of hydroxypropyl methylcellulose in an organic solvent or mixture of organic solvents
wherein the immediate release film coated tablet comprises 95% to 99% by weight of imatinib mesylate based on total weight of the film coated tablet.

6. The process of claim 5, wherein the imatinib mesylate is in alpha crystalline form characterized by a powder X-ray diffraction (PXRD) pattern having peaks at following 2θ angle positions at about 4.9, 10.5, 14.9, 16.5, 17.7, 18.1, 18.6, 19.1, 21.3, 21.6, 23.2, 24.9, 28.0, and 28.6±0.2 degrees.

7. The process of claim 6, wherein said alpha crystalline form of imatinib mesylate is retained upon storage at 40° C. and at 75% relative humidity for 3 months, when measured by an XRPD technique.

8. The process of claim 5, wherein said mesylate salt is in amorphous form.

9. The process of claim 5, wherein said tablet core consists of imatinib mesylate and a lubricant.

10. The process of claim 5, wherein the composition of the film-coated tablet is 0.10-1.00% by weight of magnesium stearate, 0.75-1.25% by weight of hydroxypropyl methylcellulose and 97.75-99% by weight of imatinib mesylate.

11. The process of claim 5, wherein said organic solvent or mixture of organic solvents is selected from the group consisting of methanol, ethanol, isopropanol, ethyl acetate, ethyl lactate, acetone, methylene chloride, 1,1,1 trichloroethane, chloroform, and mixtures thereof.

12. The process of claim 11, wherein the organic solvent is a mixture of C₁-C₃ alcohol/methylene chloride.

13. The process of claim 5, wherein said organic solvent or mixture of organic solvents is anhydrous.

14. The process of claim 5, wherein the tablet core is prepared by direct compression of alpha crystalline imtanib mesylate or amorphous imatinib mesylate, together with the one or more pharmaceutically acceptable excipients or carriers.

15. An immediate release film coated tablet prepared according to the process of claim 5.
